(19) United States
(12) Patent Application Publication
(10) Pub. No.: US 2024/0016815 A1
(43) Pub. Date: Jan. 18, 2024

(54) COMPOSITIONS AND METHODS FOR MODULATING CANCER IN NON-HUMAN MAMMALS

(71) Applicant: HOFFMAN TECHNOLOGIES LLC, New York, NY (US)

(72) Inventor: Steven HOFFMAN, Henderson, NV (US)

(21) Appl. No.: 18/255,214

(22) PCT Filed: Dec. 2, 2021
(86) PCT No.: PCT/US2021/061598

§ 371 Date: May 31, 2023

Related U.S. Application Data
(60) Provisional application No. 63/120,634, filed on Dec. 2, 2020.

(51) Int. Cl. A61K 31/568; A61K 31/407; A61K 31/198; A61K 38/12; A61K 31/4166; A61P 35/00

(52) U.S. Cl. CPC A61K 31/568; A61K 31/407; A61K 31/198; A61K 38/12; A61K 31/4166; A61P 35/00

(57) ABSTRACT

The present inventions provide compositions and methods for modulating tumors in non-human mammals using a tyrosine derivative.

### CROSS-REFERENCE TO RELATED APPLICATION

This application claims the benefit of U.S. Provisional Application No. 63/120,634, filed on Dec. 2, 2020, the entirety of which is incorporated by reference herein.

### TECHNICAL FIELD

The present inventions relate generally to methods for modulating cancer in non-human mammals, and in particular for inhibiting tumor growth and metastasis.

### BACKGROUND

The American Veterinary Medical Association estimates that 1 in 4 dogs will develop a neoplasm during the course of its lifetime, and over half of dogs over 10 years old will develop cancer. See https://www.avma.org/resources/pet-owners/petcare/cancer-pets. The most common cancers in dogs are skin cancer, breast cancer, bone cancer, leukemia, mouth cancer, lymphoma, testicular cancer, and abdominal tumors. See https://www.cancerquest.org/cancer-biology/cancer-domesticated-animals.

Domestic cats are also susceptible to cancer. The most common cancers in cats are lymphoma, squamous cell carcinoma (skin cancer), mammary cancer, mast cell tumors, oral tumors, fibrosarcoma (soft tissue cancer), osteosarcoma (bone cancer), respiratory carcinoma, intestinal adenocarcinoma, and pancreatic/liver adenocarcinoma. Id.

Livestock are also susceptible to cancers, particularly eye and skin cancers.

Treatment of cancers in animals includes surgery, radiation, and chemotherapy. None of these treatments inhibits cancer development. Generally speaking, development of cancer is a multistep process by which cells acquire genetic mutations, lose the ability to control proliferation, invade surrounding tissues and metastasize. This process, sometimes referred to as carcinogenesis, has been characterized as proceeding through four stages: initiation, promotion, progression, and conversion. See, e.g., McKinnell, R. G. et al., The Biological Basis of Cancer, Cambridge: Cambridge University Press, 1998, pages 79-81.

Initiation refers to the acquisition of a genetic mutation. Genetic mutations can be caused by exposure of a cell to, for example, carcinogens (e.g. certain chemicals, ionizing radiation), or exposure to certain viruses.

In the promotion stage, initiated cells proliferate to form benign tumors or hyperplastic lesions.

In the progression stage, the cells acquire additional genetic mutations, such as through carcinogen exposure, which allow the cells to express a neoplastic phenotype.

Conversion, or malignant transformation, refers to the acquisition of the malignant phenotype, by which the transformed cells invade surround tissues and spread.

There is a great unmet need for methods to slow or halt the carcinogenesis process, and thereby slow or halt the conversion of precancerous cells into malignant cancer. Slowing the carcinogenesis process would provide more time for the application of traditional therapeutic interventions.

### SUMMARY

The disclosure is directed to methods for treating a non-human mammal that has a benign tumor or a malignant tumor, comprising administering to the mammal an amount of at least one tyrosine derivative that is effective to modulate the tumor.

In other aspects, the disclosure is directed to methods for treating a non-human mammal that has a benign tumor or a malignant tumor, comprising administering to the mammal an amount of a combination of at least one tyrosine derivative and one or more of: (a) melanin, a melanin promoter, or a combination thereof; (b) at least one p450 3A4 promoter; (c) a leucine aminopeptidase inhibitor; (d) growth hormone inhibitor; and (e) testosterone or a derivative thereof; wherein said amount is effective to modulate said tumor.

### BRIEF DESCRIPTION OF THE DRAWINGS

FIG. 1 shows the HCT-116 tumor volume (y-axis; mm³, group average) vs. time (x-axis; days) in treated and untreated groups. The HCT116 tumor bearing athymic nude mice were PO dosed daily for 29 days at 5 ml/kg with water (Group 1), α-methyl-DL-tyrosine at 81 mg/kg (Group 2), α-methyl-DL-tyrosine at 162 mg/kg (Group 3), and α-methyl-DL-tyrosine at 324 mg/kg (Group 4).

FIG. 2 shows the Canine Brief Pain Inventory (CBPI) questionnaire for Example 2.

FIG. 3 shows the Canine Chronic Pain Scale (Colorado State University) for Example 3.

### DETAILED DESCRIPTION OF ILLUSTRATIVE EMBODIMENTS

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment incudes from the one particular and/or to the other particular value. Similarly, when values are expressed as approximations, by use of the antecedent “about,” it is understood that the particular value forms another embodiment. All ranges are inclusive and combinable.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder. This condition, disease or disorder can be cancer.

As employed above and throughout the disclosure the term “effective amount” refers to an amount effective, at dosages, and for periods of time necessary, to achieve the desired result with respect to the relevant disorder, condition, or side effect. It will be appreciated that the effective amount of components of the present invention will vary from non-human mammal to non-human mammal not only with the particular compound, component or composition selected, the route of administration, and the ability of the components to elicit a desired result in the individual, but also with factors such as the disease state or severity of the condition to be alleviated, hormone levels, age, sex, weight of the individual, the state of being of the non-human mammal, and the severity of the pathological condition, concurrent medication or special diets then being followed by the particular non-human mammal, and other factors which those skilled in the art will recognize, with the appropriate dosage being at the discretion of the attending physician. Dosage regimes may be adjusted to provide the improved therapeutic response. An effective amount is also one in which any toxic or detrimental effects of the components are outweighed by the therapeutically beneficial effects.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

Within the present invention, the disclosed compounds may be prepared in the form of pharmaceutically acceptable salts. “Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

Compounds described herein can be prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both amino and carboxy groups often exist in equilibrium with their zwitterionic forms. Thus, any of the compounds described herein that contain, for example, both amino and carboxy groups, also include reference to their corresponding zwitterions.

The term “stereoisomers” refers to compounds that have identical chemical constitution, but differ as regards the arrangement of the atoms or groups in space.

The term “inhibitor” as used herein includes compounds that inhibit the expression or activity of a protein, polypeptide or enzyme and does not necessarily mean complete inhibition of expression and/or activity. Rather, the inhibition includes inhibition of the expression and/or activity of a protein, polypeptide or enzyme to an extent, and for a time, sufficient to produce the desired effect.

The term “promoter” as used herein includes compounds that promote the expression or activity of a protein, polypeptide or enzyme and does not necessarily mean complete promotion of expression and/or activity. Rather, the promotion includes promotion of the expression and/or activity of a protein, polypeptide or enzyme to an extent, and for a time, sufficient to produce the desired effect.

The term “administering” means either directly administering a compound or composition of the present invention, or administering a prodrug, derivative or analog which will form an equivalent amount of the active compound or substance within the body.

The terms “non-human mammal,” as used herein, refers to a mammal other than a human being.

While not intending to be bound by any particular mechanism of operation, the tyrosine derivative according to the present invention function by accumulating in cancer cells and preventing them from forming a coating of either lipids or hyaluronan. By preventing the cancer cells from forming a coating of either lipids or hyaluron, the cancer cells are believed to be made more accessible to oxidative stress. Representative tyrosine derivatives include tyrosine derivatives which typically are rapidly absorbed by most cancers and inflamed tissues.

In some aspects, the disclosure is directed to methods for treating a non-human mammal that has a benign tumor or a malignant tumor, comprising administering to the mammal an amount of at least one tyrosine derivative that is effective to modulate said tumor.

In other aspects, the disclosure is directed to methods for treating a non-human mammal that has a benign tumor or a malignant tumor, comprising administering to said mammal an amount of a combination of at least one tyrosine derivative and one or more of: (a) melanin, a melanin promoter, or a combination thereof; (b) at least one p450 3A4 promoter; (c) a leucine aminopeptidase inhibitor; (d) growth hormone inhibitor; and (e) testosterone or a derivative thereof; wherein said amount is effective to modulate said tumor.

In some embodiments, modulating the tumor results in alleviating pain associated with the tumor. Thus, in some embodiments, the disclosure is directed to methods of reducing or eliminating pain caused a tumor in a non-human mammal. Methods of determining whether a reduction of pain has occurred in a mammal are known in the art, and include, for example, the Canine Brief Pain Inventory (CBPI), see Brown, D. C. et al., A Novel Approach to the Use of Animals in Studies of Pain: Validation of the Canine Brief Pain Inventory in Canine Bone Cancer, Pain Med. 10(1), 2009: 133-142, and the Canine Chronic Pain Scale (Colorado State University).

In some embodiments of the methods of the disclosure, the non-human mammal is a dog, cat, horse, cow, pig, monkey, rabbit, lamb, mouse, or goat.

In some embodiments, the non-human mammal is a dog.

In some embodiments, the non-human mammal is a cat.

In some embodiments, the non-human mammal is a horse.

In some embodiments, the non-human mammal is a cow.

In some embodiments, the non-human mammal is a pig.

In some embodiments, the non-human mammal is a monkey.

In some embodiments, the non-human mammal is a rabbit.

In some embodiments, the non-human mammal is a lamb.

In some embodiments, the non-human mammal is a mouse.

In some embodiments, the non-human mammal is a goat.

In some aspects, the disclosed methods are directed to treating a non-human mammal having a benign tumor. As used herein, the term “benign tumor” refers to tumor that is not malignant.

In some embodiments, the benign tumor is a mass of precancerous cells.

In some embodiments, the benign tumor is a mass of precancerous skin cells.

In some embodiments, the benign tumor is a mass of precancerous cells of the respiratory tract, preferably, cells of the nasal cavity, paranasal sinuses, pharynx, larynx; trachea, bronchi, bronchioles, or lungs.

In some embodiments, the benign tumor is a mass of precancerous cells of the digestive tract, preferably, cells from mouth, throat; esophagus; stomach; small intestine; colon; rectum, or anus.

In some embodiments, the benign tumor is a mass of precancerous cells of the genitourinary tract organs, preferably, cells from kidney, bladder, prostate, testicles, uterus, fallopian tubes, ovaries, vagina, or external genitalia.

In some embodiments, the benign tumor is a mass of precancerous blood cells.

In some embodiments, the benign tumor is a mass of precancerous breast cells.

In some embodiments, the benign tumor is a mass of precancerous cells of the endocrine organs, preferably, cells of the hypothalamus; pineal gland; pituitary gland; thyroid; parathyroid; thymus; adrenal gland, or pancreas.

In some embodiments, the benign tumor is a mass of precancerous brain cells.

In some aspects, the disclosed methods are directed to treating a non-human mammal having a malignant tumor. As used herein, the term “malignant tumor” refers to tumor that is malignant—i.e., that has invaded the surrounding tissues and spread.

In some embodiments, the malignant tumor is a respiratory tract tumor, preferably, of the nasal cavity, paranasal sinuses, pharynx, larynx; trachea, bronchi, bronchioles, or lungs.

In some embodiments, the malignant tumor is a digestive tract tumor, preferably, of the mouth, throat; esophagus; stomach; small intestine; colon; rectum, or anus.

In some embodiments, the malignant tumor is a genitourinary tract tumor, preferably, of the kidney, bladder, prostate, testicles, uterus, fallopian tubes, ovaries, vagina, or external genitalia.

In some embodiments, the malignant tumor is cancerous blood cells.

In some embodiments, the malignant tumor is breast tumor.

In some embodiments, the malignant tumor is an endocrine tumor, preferably, of the hypothalamus; pineal gland; pituitary gland; thyroid; parathyroid; thymus; adrenal gland, or pancreas.

In some embodiments, the malignant tumor is apocrine gland adenocarcinoma of the anal sac, hemangiosarcoma, lymphoma, mammary tumor, mast cell tumor, melanoma, oral squamous cell carcinoma, osteosarcoma, appendicular osteosarcoma, soft tissue sarcoma, solar induced squamous cell carcinoma, transitional cell carcinoma, a vaccine-associated sarcoma, adrenal medullary tumor, apocrine gland tumor, chondrosarcoma, esophageal cancer, exocrine pancreatic cancer, gastric cancer, hemangiosarcoma, hepatobiliary tumor, hyperadrenocorticism, intestinal tumor, intracranial neoplasia, larynx and trachea cancer, lymphoid leukemia, malignant histiocytoma, myelodysplasia, acute myeloid leukemia, myeloproliferative diseases, nasal chondrosarcoma, nasosinal tumor, nerve sheath tumor, ovarian tumor, plasma cell neoplas, prostate cancer, pulmonary tumors, rhabdomyosarcoma, salivary gland cancer, sebaceous and modified sebaceous gland tumor, spinal cord neoplasia, testicular tumor, thyroid gland neoplasia, urinary bladder cancer, uterine tumor, vaginal tumor, or vulvar tumor.

In some aspects, the methods of the disclosure comprise administering to the non-human mammal an amount of at least one tyrosine derivative that is effective to modulate the tumor. In other aspects, the methods of the disclosure comprise administering to the non-human mammal an amount of a combination of at least one tyrosine derivative and other compounds that is effective to modulate the tumor. As used herein, the term “modulate” refers to altering the natural course of the tumor's progression.

In some embodiments of the methods of the disclosure, the tyrosine derivatives (or combinations of agents comprising the tyrosine derivative) are effective to modulate the tumor by reducing cell proliferation in the tumor.

In some embodiments of the methods of the disclosure, the tyrosine derivatives (or combinations of agents comprising the tyrosine derivative) are effective to modulate the tumor by reducing the rate of tumor growth. As used herein, the term “reducing the rate of tumor growth” refers to decreasing the rate at which the tumor increases in size. The rate at which a tumor is growing can be determined by measuring the size of the tumor over time, and thereby determining the change in tumor size as a function of time. Administration of a tyrosine derivative, or a combination of the present disclosure, has reduced the rate of tumor growth when the change in tumor size as a function of time is less after administration of the tyrosine derivative or combination than the change in tumor size as a function of time in the absence of administration of a tyrosine derivative.

In some embodiments of the methods of the disclosure, the tyrosine derivative (or combinations of agents comprising the tyrosine derivative) is effective to modulate a benign tumor by inhibiting malignant transformation. As used herein, the term “inhibiting malignant transformation” refers to reducing the rate at which precancerous cells of the benign tumor acquire a malignant phenotype. The rate at which precancerous cells acquire a malignant phenotype can be determined by measuring the population average rate of progression from observation of the precancerous cells to malignant transformation of those cells.

As used herein, the term “malignant transformation” refers to the acquisition by cells of a malignant phenotype. A malignant phenotype is characterized by the ability of a cell to invade surrounding tissues and metastasize.

As used herein, “precancerous cells” refer to abnormal cells that are at increased risk of undergoing malignant transformation (i.e., of becoming cancerous cells). Precancerous cells are sometimes referred to as precancerous lesions.

In some embodiments of the methods of the disclosure, the tyrosine derivative (or combinations of agents comprising the tyrosine derivative) is effective to modulate a malignant tumor by inhibiting metastasis. As used herein, the term “inhibiting metastasis” refers to reducing the number of cancer cells which depart the tissue or organ in which the cancer began (i.e., the primary tumor), and spread to another site within the mammal's body.

In some embodiments of the methods of the disclosure, the tyrosine derivative (or combinations of agents comprising the tyrosine derivative) is effective to modulate a malignant tumor by reducing the number of circulating metastatic seed cells in the non-human mammal. As used herein, “circulating metastatic seed cells” refers to cancer cells that have departed a tumor and are circulating in the systemic circulation or in the lymphatic circulation. Reduction in the number of circulating metastatic seed cells can be measured by comparing the number of circulating metastatic seed cells before administering the tyrosine derivative (or combinations of agents comprising the tyrosine derivative) to the number of circulating metastatic seed cells after administering the tyrosine derivative (or combinations of agents comprising the tyrosine derivative). Methods of measuring circulating metastatic seed cells are known in the art.

In some embodiments, the circulating metastatic seed cells are in the systemic circulation.

In other embodiments, the circulating metastatic seed cells are in the lymphatic circulation.

In some embodiments, the circulating metastatic seed cells are cells of apocrine gland adenocarcinoma of the anal sac, hemangiosarcoma, lymphoma, mammary tumor, mast cell tumor, melanoma, oral squamous cell carcinoma, osteosarcoma, appendicular osteosarcoma, soft tissue sarcoma, solar induced squamous cell carcinoma, transitional cell carcinoma, a vaccine-associated sarcoma, adrenal medullary tumor, apocrine gland tumor, chondrosarcoma, esophageal cancer, exocrine pancreatic cancer, gastric cancer, hemangiosarcoma, hepatobiliary tumor, hyperadrenocorticism, intestinal tumor, intracranial neoplasia, larynx and trachea cancer, lymphoid leukemia, malignant histiocytoma, myelodysplasia, acute myeloid leukemia, myeloproliferative diseases, nasal chondrosarcoma, nasosinal tumor, nerve sheath tumor, ovarian tumor, plasma cell neoplas, prostate cancer, pulmonary tumors, rhabdomyosarcoma, salivary gland cancer, sebaceous and modified sebaceous gland tumor, spinal cord neoplasia, testicular tumor, thyroid gland neoplasia, urinary bladder cancer, uterine tumor, vaginal tumor, or vulvar tumor.

In some aspects, the present invention is directed to methods of reducing the risk of developing cancer in a non-human mammal at increased risk of developing cancer as a result of exposure to a carcinogen or an oncovirus, comprising administering to the non-human mammal an effective amount of a tyrosine derivative (or combinations of agents comprising the tyrosine derivative). In these aspects, an effective amount of a tyrosine derivative (or combination of agents comprising the tyrosine derivative) is an amount effective reduce the risk of developing cancer in the non-human mammal at increased risk of developing cancer as a result of exposure to a carcinogen or an oncovirus.

The reduction in the risk of developing cancer can be measured by comparing the mammal's risk of developing cancer before administration of the tyrosine derivative (or combinations of agents comprising the tyrosine derivative) with the mammal's risk of developing cancer after administration of the tyrosine derivative (or combinations of agents comprising the tyrosine derivative).

As used herein, the term “carcinogen” refers to a chemical substance or radiation that promotes carcinogenesis, i.e., the formation of cancer. In some embodiments, the carcinogen is a chemical substance. In other embodiments, the carcinogen is radiation.

As used herein, the term “oncovirus” refers to a virus that can cause cancer.

In all methods of the invention, the non-human mammal is administered a tyrosine derivative. In some embodiments, the tyrosine derivative is one or more of methyl (2R)-2-amino-3-(2-chloro-4 hydroxyphenyl) propanoate, D-tyrosine ethyl ester hydrochloride, methyl (2R)-2-amino-3-(2,6-dichloro-3,4-dimethoxyphenyl) propanoate H-D-Tyr(TBU)-allyl ester HCl, methyl (2R)-2-amino-3-(3-chloro-4,5-dimethoxyphenyl) propanoate, methyl (2R)-2-amino-3-(2-chloro-3-hydroxy-4-methoxyphenyl) propanoate, methyl (2R)-2-amino-3-(4-[(2-chloro-6-fluorophenyl) methoxy] phenyl) propanoate, methyl (2R)-2-amino-3-(2-chloro-3,4-dimethoxyphenyl) propanoate, methyl (2R)-2-amino-3-(3-chloro-5-fluoro-4-hydroxyphenyl) propanoate, diethyl 2-(acetylamino)-2-(4-[(2-chloro-6-fluorobenzyl) oxy] benzyl malonate, methyl (2R)-2-amino-3-(3-chloro-4-methoxyphenyl) propanoate, methyl (2R)-2-amino-3-(3-chloro-4-hydroxy-5-methoxyphenyl) propanoate, methyl (2R)-2-amino-3-(2,6-dichloro-3-hydroxy-4-methoxyphenyl) propanoate, methyl (2R)-2-amino-3-(3-chloro-4-hydroxyphenyl) propanoate, H-DL-tyr-OMe HCl, H-3,5-diiodo-tyr-OMe HCl, H-D-3,5-diiodo-tyr-OMe HCl, H-D-tyr-OMe HCl, D-tyrosine methyl ester hydrochloride, D-tyrosine-OMe HCl, methyl D-tyrosinate hydrochloride, HD-tyr-OMe·HCl, D-tyrosine methyl ester HCl, H-D-Tyr-OMe-HCl, (2R)-2-amino-3-(4-hydroxyphenyl) propionic acid, (2R)-2-amino-3-(4-hydroxyphenyl) methyl ester hydrochloride, methyl (2R)-2-amino-3-(4-hydroxyphenyl) propanoate hydrochloride, methyl (2R)-2-azanyl-3-(4-hydroxyphenyl) propanoate hydrochloride, 3-chloro-L-tyrosine, 3-nitro-L-tyrosine, 3-nitro-L-tyrosine ethyl ester hydrochloride, DL-m-tyrosine, DL-o-tyrosine, Boc-Tyr (3,542)-OSu, Fmoc-tyr(3-NO2)-0H, α-methyl-D-tyrosine, α-methyl-L-tyrosine, α-methyl-DL-tyrosine, and C₁-C₁₂ alkylester salts of α-methyl-DL-tyrosine such as α-methyl-DL-tyrosine methyl ester hydrochloride.

In some embodiments of the methods of the invention, the tyrosine derivative is α-methyl-DL-tyrosine.

In some embodiments, the methods of the invention comprise administering to said mammal (in combination with the at least one tyrosine derivative) at least one of: (a) melanin, a melanin promoter, or a combination thereof; (b) at least one p450 3A4 promoter; (c) a leucine aminopeptidase inhibitor; (d) growth hormone inhibitor; and (e) testosterone or a derivative thereof.

Suitable methods include simultaneous or at least contemporaneous administration of at least two of: the tyrosine derivative; melanin or a melanin promoter; p450 3A4 promoter; leucine aminopeptidase inhibitor; growth hormone inhibitor; and testosterone or a derivative thereof at least three of them, at least four of them, at least five of them, or each of them. It is believed to be desirable that an effective concentration of these moieties be in the subject's bloodstream at the same time, and any dosing regimen that achieves this is within the scope of the present invention. The desired number of inhibitors and promoters can be provided in a single dosage form or any number of desired dosage forms, including in individual dosage forms. Representative dosage forms include tablets, capsules, caplets, sterile aqueous or organic solutions, reconstitutable powders, elixirs, liquids, colloidal or other types of suspensions, emulsions, beads, beadlets, granules, microparticles, nanoparticles, and combinations thereof. The amount of composition administered will, of course, be dependent on the subject being treated, the subject's weight, the severity of the condition being treated, the manner of administration, and the judgment of the prescribing physician.

In some embodiments of the methods of the invention, the non-human mammal at least one of melanin, a melanin promoter, or a combination thereof. Thus, melanin can be used, one or more melanin promoters can be used, and both melanin and one or more melanin promoters can be used (either in separate dosage forms or in the same dosage form). Melanin promoters according to the present invention are chemical compounds that increase the production and/or the activity of melanin. Increased melanin levels are believed to reduce inflammation (through, for example, suppression of TNF) and exclude the sequestered lymph system. Melanin is a photo catalyst, and can therefore promote chemical reactions that generate free radicals which, in turn, can become accessible to cancer cells. Representative melanin promoters are methoxsalen, melanotan, and melanotan II.

In some embodiments of the methods, the non-human mammal is administered melanin, methoxsalen, melanotan, or melanotan II.

In some embodiments, the non-human mammal is administered melanotan.

In some embodiments, the non-human mammal is administered melanotan II.

In some embodiments of the methods of the invention, the non-human mammal is administered a p450 3A4 promoter. “Cytochrome p450 3A4” (which can be abbreviated as “p450 3A4”) is a member of the cytochrome p450 superfamily of enzymes, and is a mixed-function oxidase that is involved in the metabolism of xenobiotics in the body. It has the widest range of substrates of all of the cytochromes. The function of a p450 3A4 promoter in the methods of the invention is to increase the expression and/or the activity of p450 3A4. The increased p450 3A4 expression and/or activity is believed to reduce cortisone and estrogen levels in the patient. Additionally, the increased p450 3A4 expression and/or activity also slightly decreases blood pH, which is believed to help to preserve or enhance melanin activity. Representative p450 3A4 promoters are 5,5-diphenylhydantoin (sold commercially as, for example, Dilantin), valproic acid, and carbamazepine.

In some embodiments of the methods, the non-human mammal is administered 5,5-diphenylhydantoin (also known as phenytoin; sold commercially as, for example, Dilantin), valproic acid, or carbamazepine.

In some embodiments, the non-human mammal is administered 5,5-diphenylhydantoin.

In some embodiments of the methods, the non-human mammal a leucine aminopeptidase inhibitors (alternatively known as leucyl aminopeptidase inhibitors). Leucine aminopeptidases are enzymes that preferentially catalyze the hydrolysis of leucine residues at the N-terminus of peptides and/or proteins. Inhibiting the expression and/or activity of leucine aminopeptidases is believed to assist in tumor reabsorption by increasing cholesterol transport to the liver. Generally, it is believed that aminopeptidase inhibitors, deplete sensitive tumor cells of specific amino acids by preventing protein recycling, thus generating an antiproliferative effect. Representative leucine aminopeptidase inhibitors are N-[(2S,3R)-3-amino-2-hydroxy-4-phenylbutyryl]-L-leucine (i.e., Bestatin), rapamycin, and everolimus.

In some embodiments of the methods, the non-human mammal is administered N-[(2S,3R)-3-amino-2-hydroxy-4-phenylbutyryl]-L-leucine, rapamycin, or everolimus.

In some embodiments of the methods, the non-human mammal is administered N-[(2S,3R)-3-amino-2-hydroxy-4-phenylbutyryl]-L-leucine.

In other embodiments of the methods, the non-human mammal is administered rapamycin.

In other embodiments of the methods, the non-human mammal is administered everolimus.

In some embodiments of the methods, the non-human mammal is administered a growth hormone inhibitor. Growth hormone (such as, for example, pancreatic growth hormone) induces cell replication. Inhibition of the expression and/or activity of growth hormone is believed to exclude normal cells from rapid replication while allowing cancer cells to continue to rapidly replicate and incorporate the tyrosine derivative. Representative growth hormone inhibitors are octreotide, somatostatin, and seglitide.

In some embodiments of the methods, the non-human mammal is administered testosterone or a derivative thereof. In some embodiments, the non-human mammal is administered dihydrotestosterone.

In some embodiments, the methods of the invention comprise administering to the non-human mammal a combination of agents comprising a tyrosine derivative; at least one of melanin, a melanin promoter, or a combination thereof; a p450 3A4 promoter; and a leucine aminopeptidase inhibitor.

In some embodiments, the methods of the invention comprise administering to the non-human mammal a combination of agents comprising a tyrosine derivative; at least one of melanin, a melanin promoter, or a combination thereof; at least one of 5,5-diphenylhydantoin, valproic acid, or carbamazepine; and a leucine aminopeptidase inhibitor.

In some embodiments, the methods of the invention comprise administering to the non-human mammal a combination of agents comprising a tyrosine derivative; at least one of melanin, a melanin promoter, or a combination thereof; at least one of valproic acid, or carbamazepine; and at least one of N-[(2S,3R)-3-amino-2-hydroxy-4-phenylbutyryl]-L-leucine, rapamycin, or everolimus.

In some embodiments, the methods of the invention comprise administering to the non-human mammal a combination of agents comprising α-methyl-DL-tyrosine; at least one of melanin, a melanin promoter, or a combination thereof; at least one of valproic acid, or carbamazepine; and at least one of N-[(2S,3R)-3-amino-2-hydroxy-4-phenylbutyryl]-L-leucine, rapamycin, or everolimus.

In some embodiments, the methods of the invention comprise administering to the non-human mammal a combination of agents comprising α-methyl-DL-tyrosine, melanotan, 5,5-diphenylhydantoin, and N-[(2S,3R)-3-amino-2-hydroxy-4-phenylbutyryl]-L-leucine.

In other embodiments, the methods of the invention comprise administering to the non-human mammal a combination of agents comprising α-methyl-DL-tyrosine, melanotan II, 5,5-diphenylhydantoin, and N-[(2S,3R)-3-amino-2-hydroxy-4-phenylbutyryl]-L-leucine.

In some embodiments, the methods of the invention comprise administering to the non-human mammal a combination of agents comprising α-methyl-DL-tyrosine, melanotan, 5,5-diphenylhydantoin, and rapamycin.

In other embodiments, the methods of the invention comprise administering to the non-human mammal a combination of agents comprising α-methyl-DL-tyrosine, melanotan II, 5,5-diphenylhydantoin, and rapamycin.

In some embodiments, the methods of the invention comprise administering to the non-human mammal a combination of agents comprising α-methyl-DL-tyrosine, melanotan, 5,5-diphenylhydantoin, N-[(2S,3R)-3-amino-2-hydroxy-4-phenylbutyryl]-L-leucine, and testosterone.

In other embodiments, the methods of the invention comprise administering to the non-human mammal a combination of agents comprising α-methyl-DL-tyrosine, melanotan II, 5,5-diphenylhydantoin, N-[(2S,3R)-3-amino-2-hydroxy-4-phenylbutyryl]-L-leucine, and testosterone.

In some embodiments, the methods of the invention comprise administering to the non-human mammal a combination of agents comprising α-methyl-DL-tyrosine, melanotan, 5,5-diphenylhydantoin, rapamycin, and testosterone.

In other embodiments, the methods of the invention comprise administering to the non-human mammal a combination of agents comprising α-methyl-DL-tyrosine, melanotan II, 5,5-diphenylhydantoin, rapamycin, and testosterone.

In some embodiments, the methods of the invention comprise administering to the non-human mammal a combination of agents comprising α-methyl-DL-tyrosine, melanotan, 5,5-diphenylhydantoin, N-[(2S,3R)-3-amino-2-hydroxy-4-phenylbutyryl]-L-leucine, and dihydrotestosterone.

In other embodiments, the methods of the invention comprise administering to the non-human mammal a combination of agents comprising α-methyl-DL-tyrosine, melanotan II, 5,5-diphenylhydantoin, N-[(2S,3R)-3-amino-2-hydroxy-4-phenylbutyryl]-L-leucine, and dihydrotestosterone.

In some embodiments, the methods of the invention comprise administering to the non-human mammal a combination of agents comprising α-methyl-DL-tyrosine, melanotan, 5,5-diphenylhydantoin, rapamycin, and dihydrotestosterone.

In other embodiments, the methods of the invention comprise administering to the non-human mammal a combination of agents comprising α-methyl-DL-tyrosine, melanotan II, 5,5-diphenylhydantoin, rapamycin, and dihydrotestosterone.

In the methods of the present invention, the tyrosine derivative may be administered to the non-human mammal through any suitable administration route, including for example, orally, perorally, nasally, subcutaneously, intravenously, intramuscularly, transdermally, vaginally, rectally or in any combination thereof. In embodiments of the methods that comprise administering (a) melanin, a melanin promoter, or a combination thereof; (b) at least one p450 3A4 promoter; (c) leucine aminopeptidase inhibitor; (d) a growth hormone inhibitor; and/or (e) testosterone or a derivative thereof; the (a) melanin, a melanin promoter, or a combination thereof; (b) at least one p450 3A4 promoter; (c) leucine aminopeptidase inhibitor; (d) a growth hormone inhibitor; and/or (e) testosterone or a derivative thereof may be administered to the non-human mammal through any suitable administration route, including for example, orally, perorally, nasally, subcutaneously, intravenously, intramuscularly, transdermally, vaginally, rectally or in any combination thereof.

In some embodiments, the tyrosine derivatives are administered in a pharmaceutical composition comprising the tyrosine derivative and a pharmaceutically acceptable excipient. Pharmaceutically acceptable excipients are known to those skilled in the art. See, e.g., Handbook of Pharmaceutical Excipients (Rowe, ed.), Pharmaceutical Press; 6th Revised edition (Jul. 31, 2009).

In embodiments of the methods wherein the tyrosine derivatives are administered in combination with (a) melanin, a melanin promoter, or a combination thereof; (b) at least one p450 3A4 promoter; (c) leucine aminopeptidase inhibitor; (d) a growth hormone inhibitor; and/or (e) testosterone or a derivative thereof; the (a) melanin, a melanin promoter, or a combination thereof; (b) at least one p450 3A4 promoter; (c) leucine aminopeptidase inhibitor; (d) a growth hormone inhibitor; and/or (e) testosterone or a derivative thereof may be administered together with the tyrosine derivative in the same pharmaceutical composition, or may be administered in one or more separate pharmaceutical compositions.

In some embodiments, pharmaceutical compositions used in the methods of the invention can further include D-leucine. D-leucine is a stereoisomer of the naturally occurring L-leucine, the form of leucine incorporated into polypeptides and proteins. D-leucine cannot be incorporated into polypeptides and/or proteins. Along with the leucine aminopeptidase inhibitor, the D-leucine is believed to create a physiological environment that mimics a leucine shortage. Thus, the presence of D-leucine permits the use of lower doses of leucine aminopeptidase inhibitor in a pharmaceutical composition.

In some embodiments of the methods of the disclosure, the non-human mammal is further administered an amount of one or more additional therapeutic agents.

In some embodiments, the additional therapeutic agent is 5 fluorouracil, actinomycin, bleomycin, carboplatin, chlorambucil, cisplatin, cyclophosphamide, cyclophosphamine, cytosine arabinoside, doxorubicin, dacarbazine (DTIC), L-asparaginase, melphalan, methotrexate, mitoxantrone, piroxicam, prednisone, vinblastine, or vincristine.

Also provided herein are kits including a combination therapy of the present invention. Representative kits comprise a tyrosine derivative, melanin and/or a melanin promoter, a p450 3A4 promoter, a leucine aminopeptidase inhibitor, testosterone or a derivative thereof, and, optionally, a growth hormone inhibitor of the type described above, together with packaging for same. The kit can include one or more separate containers, dividers or compartments and, optionally, informational material such as instructions for administration. For example, each inhibitor or promoter (or the various combinations thereof) can be contained in a bottle, vial, or syringe, and the informational material can be contained in a plastic sleeve or packet or provided in a label. In some embodiments, the kit includes a plurality (e.g., a pack) of individual containers, each containing one or more unit dosage forms of a compound described herein. For example, the kit can include a plurality of syringes, ampules, foil packets, or blister packs, each containing a single unit dose of a compound described herein or any of the various combinations thereof. The containers of the kits can be air tight, waterproof (e.g., impermeable to changes in moisture or evaporation), and/or light-tight. The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device.

Suitable methods include simultaneous or at least contemporaneous administration of at least two of: the tyrosine derivative; melanin or a melanin promoter; p450 3A4 promoter; leucine aminopeptidase inhibitor; testosterone or derivative thereof; at least three of them, or each of them (in each case, optionally, with a growth hormone inhibitor). It is believed to be desirable that an effective concentration of these moieties be in the subject's bloodstream at the same time, and any dosing regimen that achieves this is within the scope of the present invention. The desired number of inhibitors and promoters can be provided in a single dosage form or any number of desired dosage forms, including in individual dosage forms. Representative dosage forms include tablets, capsules, caplets, sterile aqueous or organic solutions, reconstitutable powders, elixirs, liquids, colloidal or other types of suspensions, emulsions, beads, beadlets, granules, microparticles, nanoparticles, and combinations thereof. Transdermal administration can be effected using, for example, oleic acid, 1-methyl-2-pyrrolidone, or dodecylnonaoxyethylene glycol monoether. The amount of composition administered will, of course, be dependent on the subject being treated, the subject's weight, the severity of the condition being treated, the manner of administration, and the judgment of the prescribing physician.

In the methods of the invention, the administered compounds (i.e., the tyrosine derivatives, the melanin, promoters, inhibitors, and/or testosterone or derivatives thereof) can be administered during a cycle consisting of five to seven days of administering the compounds and one to two days of not administering the compounds. The compounds can be administered over the course of at least six of said cycles. It can be desirable to administer these compounds about two hours between meals to facilitate uptake.

In certain embodiments, at least two of the compounds (i.e., tyrosine derivative, melanin, promoters, inhibitors, and/or testosterone or derivatives thereof) are administered simultaneously. In other embodiments, at least three of the components are administered simultaneously. Each of the components can be administered simultaneously. In suitable embodiments, the components are administered orally, subcutaneously, intravenously, transdermally, vaginally, rectally or in any combination thereof. The transdermal administration can be done with oleic acid, 1-methyl-2-pyrrolidone, or dodecylnonaoxyethylene glycol monoether. In other embodiments, the components are administered during a cycle consisting of five to seven days of administering the components and one to two days of not administering the components. The components can be administered over the course of at least six of said cycles.

In certain embodiments of the present invention, a pharmaceutical composition or combination therapy may be administered to a non-human mammal for 5 days per week for a period of 6 weeks, creating one cycle of 30 days of treatment. Depending on the outcome after 6 weeks or one cycle of treatment, additional cycles of the pharmaceutical composition or combination therapy may be administered.

In one representative method, 60 mg of the tyrosine derivative is administered orally and 0.25 mL of a 2 mg/mL suspension of the tyrosine derivative is administered subcutaneously; 10 mg of the methoxsalen is administered orally and 0.25 mL of a 1 mg/mL suspension of the methoxsalen is administered subcutaneously; 30 mg of the 5,5-diphenylhydantoin is administered orally; and 20 mg of the N-[(2S,3R)-3-amino-2-hydroxy-4-phenylbutyryl]-L-leucine is administered orally.

In certain embodiments, the combination therapy comprises: (i) a dosage form containing melanin (50 mcg) and α-methyl-DL-tyrosine (75 mg); (ii) a dosage form containing 5,5-diphenylhydantoin (15 mg) and α-methyl-DL-tyrosine (75 mg); (iii) a dosage form containing 3-amino-2-hydroxy-4-phenylbutyryl]-L-leucine (50 mcg) and α-methyl-DL-tyrosine (75 mg); (iv) a dosage form containing 3-amino-2-hydroxy-4-phenylbutyryl]-L-leucine (5 mcg), melanotan II (10 mcg), and 5,5-diphenylhydantoin (2 mg); and (v) a dosage form containing α-methyl-DL-tyrosine (5 mg) in NaCl bacteriostatic water.

In other embodiments, the combination therapy comprises: (i) a dosage form containing melanin (50 mcg) and α-methyl-DL-tyrosine (75 mg); (ii) a dosage form containing 5,5-diphenylhydantoin (15 mg) and α-methyl-DL-tyrosine (75 mg); (iii) a dosage form containing rapamycin (0.2 mg) and α-methyl-DL-tyrosine (75 mg); (iv) a dosage form containing rapamycin (0.15 mcg), melanotan II (10 mcg), and 5,5-diphenylhydantoin (2 mg); and (v) a dosage form containing α-methyl-DL-tyrosine (5 mg) in NaCl bacteriostatic water. Dosages that are two times greater than this, and even four times greater than this, are believed to be both safe and efficacious.

In a suitable embodiment of the method, 60 mg of the tyrosine derivative is administered orally and 0.25 mL of a 2 mg/mL suspension of the tyrosine derivative is administered subcutaneously. The melanin promoter can be methoxsalen. In another suitable method, 10 mg of the methoxsalen is administered orally and 0.25 mL of a 1 mg/mL suspension of the methoxsalen is administered subcutaneously. The melanin promoter can also be melanotan II. The p450 3A4 promoter can be 5,5-diphenylhydantoin. In another suitable method, 30 mg of the 5,5-diphenylhydantoin is administered orally. The p450 3A4 promoter can also be valproic acid or carbamazepine. The leucine aminopeptidase inhibitor can be N-[(2S,3R)-3-amino-2-hydroxy-4-phenylbutyryl]-L-leucine. In another suitable method, 20 mg of the N-[(2S,3R)-3-amino-2-hydroxy-4-phenylbutyryl]-L-leucine is administered orally. The leucine aminopeptidase inhibitor can also be rapamycin. The growth hormone can be pancreatic growth hormone. The growth hormone inhibitor can be octreotide. The method can further comprise administering an effective amount of D-leucine.

In some embodiments, the combination therapy comprises α-methyl-DL-tyrosine (8 mg), Bestatin (9 mg), Dihydrotestosterone (40 μg), and Melanotan (20 μg).

In some embodiments, the combination therapy comprises α-methyl-DL-tyrosine (8 mg), Bestatin (50 Dihydrotestosterone (40 μg), and Melanotan (20 μg).

The present methods can include not only the disclosed administration step but also the step of assessing progression of said cancer in said subject and/or the extent of cellular proliferation. The assessing step can be performed before or after the administering step.

The following examples of specific embodiments for carrying out the present invention are offered for illustrative purposes only and are not intended to limit the scope of the present invention in any way.

### EXAMPLE

### Example 1

A study was conducted to evaluate the antitumor effect of an oral formulations of α-methyl-DL-tyrosine in athymic nude mice bearing HCT116 tumors.

Athymic nude mice (female, 6-7 weeks old) were purchased from Charles River Laboratories, USA. The mice were housed in a ventilated cage rack with HEAP filter system, with 12-hour light cycle at 21-24° C. (70-75° F.) and 40-60% humidity. The mice were fed with Rodent Diet 5001 (Lab Diet) consisting of: >23% protein; >4.5% fat; and >6% fiber. The mice were allowed free access to water (Distill water, 1 ppm Cl).

The mice were acclimated for 5 days prior to the implantation of HCT116 cells at 1 million cells per mouse in 100 ul of 1XPBS containing 50% Matrigel.

At the completion of study, mice were euthanized with isoflurane followed by inhalation of carbon dioxide (CO2).

The HCT116 cells (human colon carcinoma) were purchased from ATCC, and expanded to sufficient number of cells for tumor cell implantation.

Solutions of α-methyl-DL-tyrosine at 16.2 mg/ml, 32.4 mg/ml, and 64.8 mg/ml in water were prepared.

After an acclimation period of 5 days, fifty (50) athymic nude female mice were inoculated in the thigh/flank area with 1 million of HCT116 cells suspended in 100 μl 1×PBS containing 50% Matrigel.

Beginning at Day 6 post cell inoculation, the tumor volume was measured every day until the average tumor volume reached about 100 mm3 (Tumor Volume=length×width×width×0.5).

Forty-four (44) out of the fifty (50) mice within the range of 50-150 mm3 were selected and randomly grouped into 4 groups (n=11): a control group treated with water (Group 1) and three groups treated with α-methyl-DL-tyrosine at 81 mg/kg (Group 2), 162 mg/kg (Group 3), and 324 mg/kg (Group 4).

The HCT-116 tumor bearing mice were oral gavage (PO) dosed daily at 5 ml/kg with water (Group 1), α-methyl-DL-tyrosine at 81 mg/kg (Group 2), α-methyl-DL-tyrosine at 162 mg/kg (Group 3), and α-methyl-DL-tyrosine at 324 mg/kg (Group 4) for 28 days.

Clinical observations were made daily; tumor volume was measured every three to four days prior to dosing until the completion of study or when individual tumor volume exceeds 2000 mm3.

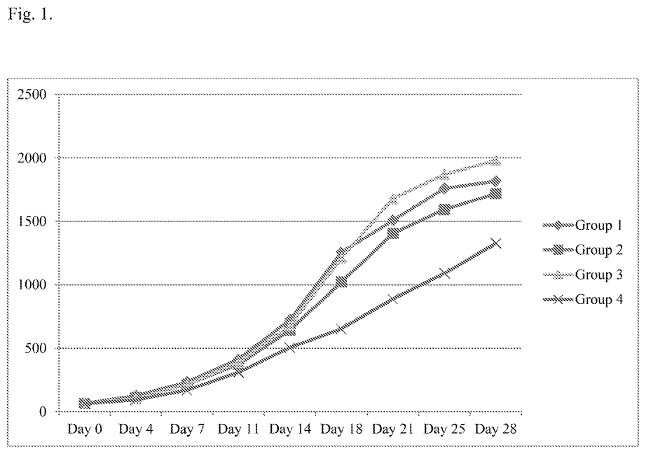

As summarized in Table 1 and presented in FIG. 1, the daily PO treatment of HCT-116 tumor bearing mice with α-methyl-DL-tyrosine at 324 mg/kg demonstrated a statistically significant (p<0.05, t-test) tumor growth inhibition.

The maximum anti-tumor efficacy was 48% reduction in tumor volume observed on Day 22.

TABLE 1
HCT-116 Tumor volume (mm³, group average) in treatment groups

| | | Day (post 1st dosing) | | | | | | | | |
|---|---|---|---|---|---|---|---|---|---|---|
| Group | | 0 | 4 | 7 | 11 | 14 | 18 | 21 | 25 | 28 |
| G1, water, | Group | 64.8 | 127.6 | 233.9 | 414.3 | 725.2 | 1255.8 | 1507.0 | 1759.5 | 1815.9 |
| n = 11 | Avg | | | | | | | | | |
| G2, α- | Group | 63.5 | 112.5 | 215.3 | 373.6 | 643.5 | 1022.3 | 1405.9 | 1592.4 | 1718.8 |
| methyl-DL- | Avg | | | | | | | | | |
| tyrosine at | | | | | | | | | | |
| 162 mg/kg, | | | | | | | | | | |
| n = 11 | | | | | | | | | | |
| G3, α- | Group | 64.3 | 106.0 | 214.6 | 391.4 | 676.6 | 1210.3 | 1680.1 | 1868.0 | 1979.2 |
| methyl-DL- | Avg | | | | | | | | | |
| tyrosine at | | | | | | | | | | |
| 81 mg/kg, | | | | | | | | | | |
| n = 11 | | | | | | | | | | |
| G4, α- | Group | 64.2 | 92.9 | 172.3 | 310.5 | 506.3 | 654.9 | 890.6 | 1090.1 | 1325.7 |
| methyl-DL- | Avg | | | | | | | | | |
| tyrosine at | | | | | | | | | | |
| 324 mg/kg, | | | | | | | | | | |
| n = 11 | | | | | | | | | | |

These data demonstrate that treatment of HCT116 tumor bearing mice with α-methyl-DL-tyrosine at 324 mg/kg daily resulted in a statistically significant (p<0.05, t-test) antitumor efficacy between Day 18 to Day 25. On Day 28, the tumor inhibition became non-significant simply because the study limited the maximum tumor volume at 2000 mm3 in the control group.

Moreover, in this study the α-methyl-DL-tyrosine was administered seven days after the tumor was implanted in the mouse. The fact that the α-methyl-DL-tyrosine inhibited growth of the tumor at the early phase of tumor implantation demonstrates that α-methyl-DL-tyrosine can inhibit metastatic conversion of precancerous cells.

### Example 2

A study is performed to assess pain management and safety of metabolomic therapy in dogs with appendicular osteosarcoma (OSA).

The effectiveness endpoints include veterinary assessment of pain on palpation and client assessment of pain, but does not objectively assess antitumor response. Canine Brief Pain Inventory (CPBI) is assessed by the owner and the investigator assesses response to palpation using the Colorado State Chronic Pain Scale. Safety is a secondary endpoint.

Osteosarcoma is a common primary bone tumor particularly in medium to giant breed dogs affecting appendicular bones such as the distal radius, proximal humerus, distal femur and proximal tibia. The osteolytic/osteoproductive behavior of this tumor results in significant pain and discomfort. Amputation +/− adjuvant chemotherapy represents the standard treatment approach though amputation may not be ideal due to co-morbidities such as osteoarthritis in other limbs. When amputation is not pursued, hypofractionated radiation therapy +/− bisphosphonate therapy may be used to help manage pain.

Dogs in this study are administered a therapeutic “cocktail” (IVP) which includes bestatin, α-methyl-DL-tyrosine (α-methylparatyrosine), dihydrotestosterone and melanotan diluted with 0.9% NaCL delivered as 3 times weekly subcutaneous (SQ) administration. Without intending to be bound by theory, this combination of these agents is proposed to alter tumor cell metabolism and thereby improve pain in dogs with appendicular OSA.

Bestatin is administered at a dose of 9.0 mg/dog in this study.

The compound α-methyl-DL-tyrosine (α-methylparatyrosine; AMPT) is administered at a dose of 8 mg/dog.

Dihydrotestosterone is administered at a dose of 40 μg/dog in this study.

Melanotan is administered at a dose 20 μg/dog.

The drug combination is provided in single use, preloaded syringes for subcutaneous administration. The syringes are stored at room temperature. The drug combination is administered as a 0.2 ml subcutaneous injection on Monday, Wednesday and Friday.

Inclusion criteria: For a dog to be enrolled in the study, all of the following inclusion criteria must be met:
Written owner informed consent has been obtained prior to screening the dog;
The dog is ≥1 yr of age;
The dog has a minimum body weight 5 kg;
Cytological or histopathological diagnosis of osteosarcoma (high suspicion of OSA on radiographs will also be permitted upon approval of ACI and the Sponsor);
The dog has gross appendicular OSA;
The dog has a Veterinary Cooperative Oncology Group (VCOG) Performance Score of 0 or 1 [0=normal activity; 1=restricted activity: decreased activity from pre-disease status];
Prior glucocorticoid therapy, chemotherapy, bisphosphonates, radiation therapy or small molecule inhibitor therapy is acceptable following a 21-day washout;
Prior immunotherapy or targeted monoclonal antibody therapy is acceptable following a 42-day washout;
Dog's CBPI cumulative score (pain severity+interference domain)≥12;
Prior use of NSAIDs are permitted granted the dog has been receiving the current regimen for at least 14 days prior to enrollment. Further, ongoing treatments should maintain the same dosage and treatment schedule as used prior to enrollment.

Exclusion criteria: If present, the following exclusion criteria will disqualify a dog from being enrolled in the study:
The dog has any underlying disease that in the opinion of the investigator and Medical Director will affect the study objectives or overall patient safety;
The dog is pregnant, lactating, or likely to become pregnant;
The dog is participating in another study;
The dog may not be available for the entire study duration.

If a dog fulfills all screening criteria, the dog is enrolled in the study. If a dog fails any of these screening/enrollment activities, the dog is not enrolled in the study, but may be re-screened at a later date.

Screening activities take place between Day −14 and Day 0 and include the following:
Discuss study proceedings with the owner, and obtain signed owner informed consent.
Collect relevant medical history.
Record all medications administered within the last 42 days.
Perform a physical examination and record the results.
Assess performance score.
Owner completion of CBPI for eligibility.
Collect blood and urine for Complete Blood Count (CBC), chemistry panel and urinalysis.
Perform radiographs of the affected limb.

Prior to treatment administration, enrollment activities take place on Day 0 and include the following:
Perform a physical examination and record the results.
Assess performance score.
Owner completion of CBPI.
Veterinarian assessment: pain on palpation.
Complete, review and finalize inclusion/exclusion form.
Instruct owner on treatment administration.
Perform treatment administration with IVP and record on the appropriate dosing CRF.

The IVP begins following or on Day 0 and is administered daily by the owner as a 0.2 mL SQ injection Monday, Wednesday and Friday until study completion.

Dogs are required to return to the study site on Day 7 (±2), Day 14 (±2) and Day 28 (±2). Owners are instructed to complete CBPI at home on Day 21 (±2).

On Day 7 (±2), the dog is re-evaluated by the investigator or examining veterinarian. The following procedures are performed:
Perform a physical examination and record the results.
Assess performance score.
Owner completion of CBPI.
Veterinarian assessment: pain on palpation.
   Record any adverse events.
Record concomitant medications.

On Day 14 (±2), the dog is re-evaluated by the investigator or examining veterinarian. The following procedures are performed:
Perform a physical examination and record the results.
Assess performance score.
Owner completion of CBPI.
Veterinarian assessment: pain on palpation.
Collect blood and urine for Complete Blood Count (CBC), chemistry panel and urinalysis.
Record any adverse events.
Record concomitant medications.

On Day 21 (±2), the following procedures are performed:
Owner completion of CBPI.

On Day 28 (±2), the dog is re-evaluated by the investigator or examining veterinarian. The following procedures are performed:
Perform a physical examination and record the results.
Assess performance score.
Owner completion of CBPI.
Veterinarian assessment: pain on palpation.
Collect blood and urine for Complete Blood Count (CBC), chemistry panel and urinalysis.
Perform radiographs of the affected limb.
Record any adverse events.
Record concomitant medications.

The medical history is collected at the Screening Visit (−14 to 0 days) before study enrollment and should include (but is not limited to) demographic information, and previous pertinent medical/surgical history for the last 42 days. Prior tumor treatments to include but not limited to surgeries in the past 180 days are collected.

A physical examination is completed by the investigator/examining veterinarian at each scheduled and unscheduled visit, or early removal from the study and results are recorded. When at all possible, all examinations should be performed by the same individual for each dog on study. The following are evaluated and abnormalities are noted:
General clinical examination (minimum): Vitals—body weight (kg), temperature (° F.), heart rate, and respiratory rate
General appearance
Eyes, ears, mouth
Cardiovascular
Respiratory
Musculoskeletal
Gastrointestinal
Integument
Neurological
Urogenital
Lymphatic

Body weights are recorded in kilograms to the nearest one tenth of a kilogram.

Clinical Pathology: Study dogs do not need to be fasted prior to the collection of blood samples. Complete blood count (CBC, including differential) and serum biochemistry specimens are collected at screening, Day 14 (±2), and Day 28 (±2) via jugular or peripheral venipuncture for analysis during the course of the study (4-6 mL collected per visit).

As soon as available, clinical pathology results are evaluated by the investigator/examining veterinarian and the clinical significance of values falling outside of the reference range are determined and explained if the results are considered clinically significant.

Complete Blood Count (CBC) At a minimum, the following hematology parameters are measured from samples taken at screening, Day 14 (±2), and Day 28 (±2), and upon early removal from the study, and, if determined necessary by the investigator, at the time of an unscheduled visit:
White blood count (WBC)
Mean corpuscular hemoglobin concentration (MCHC)
WBC Differential
Mean corpuscular volume (MCV)
Red blood count (RBC)
RBC and WBC morphology
Hematocrit (HCT)
Platelet count
Hemoglobin (HGB)
Platelet morphology

Serum Chemistry Panel: At a minimum, the following serum chemistry parameters are measured from blood samples taken at screening, Day 14 (±2), and Day 28 (±2) and upon early removal from the study, and, if determined necessary by the investigator, at the time of an unscheduled visit.
Albumin
Aspartate transaminase (AST)
Globulin
Total bilirubin
Albumin/Globulin ratio
Calcium
Total protein
Potassium
Creatinine kinase (CPK)
Chloride
Alkaline phosphatase
Sodium
Alanine aminotransferase
Sodium/Potassium Ratio
Blood urea nitrogen (BUN)
Phosphorus
Creatinine
Glucose
BUN/Creatinine Ratio
Cholesterol

Urinalysis: Urine samples for routine urinalysis are collected by the owner or veterinarian and analyzed at screening, Day 14 (±2), and Day 28 (±2) and upon early removal from the study, and, if determined necessary by the investigator, at the time of an unscheduled visit.
Urine protein
Glucose
pH
Ketones
Color
Microscopic examination of the sediment for RBC/hpf, WBC/hpf, bacteria,
epithelial cells, mucus, casts and crystals
Clarity
Blood
Bilirubin
Specific gravity measured by refractometer

Radiographs of the affected limb (minimum AP and lateral views) are performed at screening and Day 28 (±2). A radiology report is generated by the investigator, examining veterinarian, or radiologist. Additional imaging such as thoracic radiography is permitted at the discretion of the investigator.

Performance Score: At each study visit, the investigator assesses performance scores using VCOG (listed below). Only dogs with scores of 0 or 1 are enrolled in the study.
0=normal activity;
1=mild lethargy over baseline; diminished activity from predisease level, but able to function as an acceptable pet;
2=moderate lethargy causing some difficulty with performing Activities of Daily Living (ADL); ambulatory only to the point of eating, sleeping, and consistently defecating and urinating in acceptable areas;
3=compromised, severely restricted in ADL; unable to confine urinations and defecation to acceptable areas; will consume food if offered in place;
4=disabled, must be force fed and helped to perform ADL;
5=death

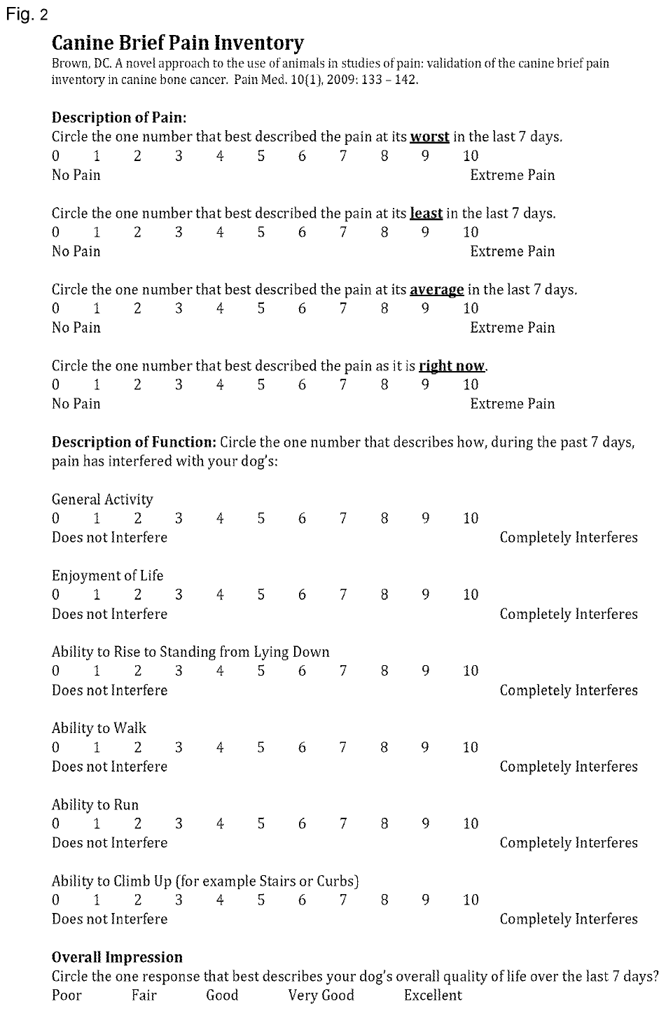

Canine Brief Pain Inventory (CBPI): Responses to the CBPI owner-completed questionnaire are used as the primary efficacy variables in this study. See Brown, D. C. et al., A Novel Approach to the Use of Animals in Studies of Pain: Validation of the Canine Brief Pain Inventory in Canine Bone Cancer, Pain Med. 10(1), 2009: 133-142. Responses to the four pain severity questions are averaged at each visit to provide a pain severity score. Changes in pain severity score are compared before and after treatment in the same dog. Similarly, responses to the six pain interference questions are averaged at each visit to provide a pain interference score. Changes in pain interference score are compared before and after treatment in the same dog. Treatment success are defined as a reduction of ≥2 in pain interference score and ≥1 in pain severity score. See FIG. 2.

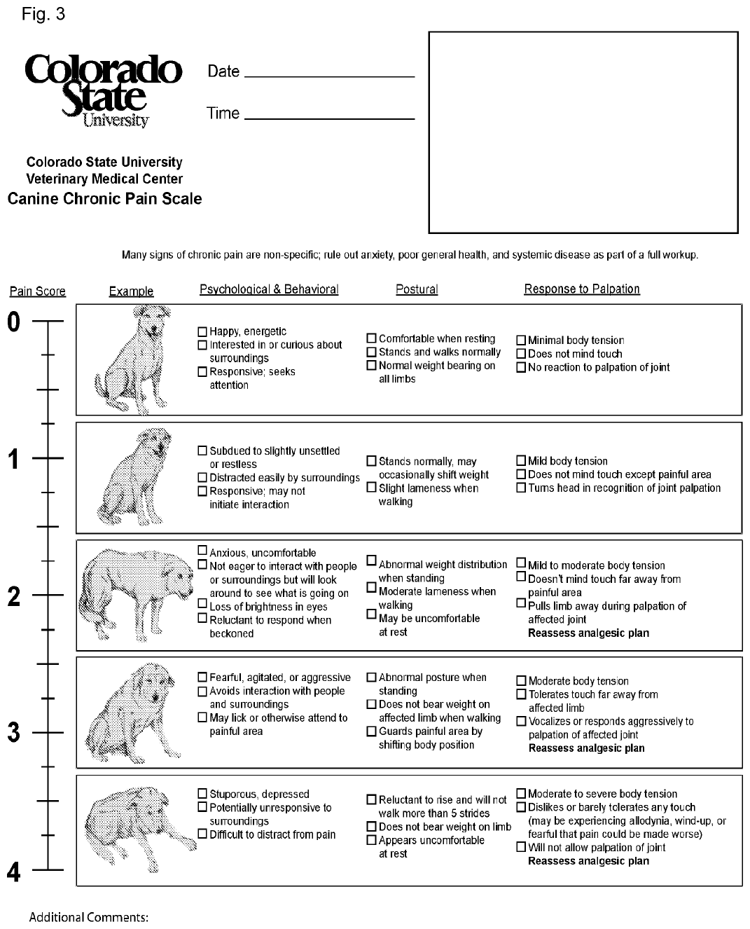

Pain on palpation: Response to palpation of the affected limb is evaluated using the section “Response to Palpation” of the Canine Chronic Pain Scale (Colorado State University). Changes in “Response to Palpation” score are compared to baseline (Day 0). Treatment success is defined as a reduction of 1 point compared to Day 0. See FIG. 3.

Statistical Methodology: This is a proof-of-concept study in client-owned dogs. Descriptive statistics (number of cases, mean, standard deviation, minimum, median and maximum values) are presented for continuous variables. No hypothesis testing is performed.

The study demonstrates that the combination of drugs is effective to either reduce pain or halt the increase in pain in dogs with OSA.

In some embodiments, the disclosure is directed to the following aspects:
Aspect 1. A method for treating a non-human mammal that has a benign tumor or a malignant tumor, comprising administering to said mammal an amount of at least one tyrosine derivative that is effective to modulate said tumor.
Aspect 2. A method for treating a non-human mammal that has a benign tumor or a malignant tumor, comprising administering to said mammal an amount of a combination of at least one tyrosine derivative and one or more of: (a) melanin, a melanin promoter, or a combination thereof; (b) at least one p450 3A4 promoter; (c) a leucine aminopeptidase inhibitor; (d) growth hormone inhibitor; and (e) testosterone or a derivative thereof; wherein said amount is effective to modulate said tumor.
Aspect 3. The method of aspect 1 or aspect 2 wherein the tyrosine derivative is one or more of:
methyl (2R)-2-amino-3-(2-chloro-4-hydroxyphenyl) propanoate; D-tyrosine ethyl ester hydrochloride;
methyl (2R)-2-amino-3-(2,6-dichloro-3,4-dimethoxyphenyl) propanoate; H-D-Tyr(TBU)-allyl ester HCl;
methyl (2R)-2-amino-3-(3-chloro-4,5-dimethoxyphenyl) propanoate;
methyl (2R)-2-amino-3-(2-chloro-3-hydroxy-4-methoxyphenyl) propanoate;
methyl (2R)-2-amino-3-(4-[(2-chloro-6-fluorophenyl) methoxy] phenyl) propanoate;
methyl (2R)-2-amino-3-(2-chloro-3,4-dimethoxyphenyl) propanoate;
methyl (2R)-2-amino-3-(3-chloro-5-fluoro-4-hydroxyphenyl) propanoate;
diethyl 2-(acetylamino)-2-(4-[(2-chloro-6-fluorobenzyl) oxy] benzyl malonate;
methyl (2R)-2-amino-3-(3-chloro-4-methoxyphenyl) propanoate;
methyl (2R)-2-amino-3-(3-chloro-4-hydroxy-5-methoxyphenyl) propanoate;
methyl (2R)-2-amino-3-(2,6-dichloro-3-hydroxy-4-methoxyphenyl) propanoate;
methyl (2R)-2-amino-3-(3-chloro-4-hydroxyphenyl) propanoate;
H-DL-tyr-OMe HCl;
H-3,5-diiodo-tyr-OME HCl;
H-D-3,5-diiodo-tyr-OME HCl;
H-D-tyr-OMe HCl;
D-tyrosine methyl ester hydrochloride;
D-tyrosine-OMe HCl;
methyl D-tyrosinate hydrochloride;
D-tyrosine methyl ester HCl;
H-D-Tyr-OMe-HCl;
(2R)-2-amino-3-(4-hydroxyphenyl) propionic acid;
(2R)-2-amino-3-(4-hydroxyphenyl) methyl ester hydrochloride;
methyl (2R)-2-amino-3-(4-hydroxyphenyl) propanoate hydrochloride;
methyl (2R)-2-azanyl-3-(4-hydroxyphenyl) propanoate hydrochloride;
3-chloro-L-tyrosine;
3-nitro-L-tyrosine;
3-nitro-L-tyrosine ethyl ester hydrochloride;
DL-m-tyrosine;
DL-o-tyrosine;
Boc-Tyr (3,5-I2)-OSu;
Fmoc-tyr(3-NO2)-OH;
α-methyl-L-tyrosine;
α-methyl-D-tyrosine;
α-methyl-DL-tyrosine; and
C₁-C₁₂ alkylester salts of α-methyl-DL-tyrosine such as α-methyl-DL-tyrosine methyl ester hydrochloride.
   Aspect 4. The method of aspect 3 wherein the tyrosine derivative is α-methyl-DL-tyrosine.
   Aspect 5. The method of any one of aspects 2-4, wherein said mammal is administered an amount of a combination of at least one tyrosine derivative and (a) melanin, a melanin promoter, or a combination thereof; (b) at least one p450 3A4 promoter; (c) a leucine aminopeptidase inhibitor; and (e) testosterone or a derivative thereof, wherein said amount is effective to modulate said tumor.
   Aspect 6. The method of aspect 5, wherein the tyrosine derivative is α-methyl-DL-tyrosine; (a) is melanin, methoxsalen, melanotan, or melanotan II; (b) is 5,5-diphenylhydantoin; (c) Bestatin, rapamycin, or everolimus, and (e) is dihydrotestosterone.
   Aspect 7. The method of aspect 6, wherein the tyrosine derivative is α-methyl-DL-tyrosine; (a) is melanotan II; (b) is 5,5-diphenylhydantoin; (c) Bestatin, and (e) is dihydrotestosterone.
   Aspect 8. The method of aspect 6, wherein the tyrosine derivative is α-methyl-DL-tyrosine; (a) is melanotan (b) is 5,5-diphenylhydantoin; (c) is Bestatin, and (e) is dihydrotestosterone.
   Aspect 9. The method of any one of aspects 1-8, wherein said amount is effective to modulate said tumor by reducing cell proliferation in the tumor.
   Aspect 10. The method of any one of aspects 1-9 wherein said amount is effective to modulate said tumor by reducing the rate of tumor growth.
   Aspect 11. The method of any one of aspects 1-10 wherein said amount is effective to modulate said tumor by inhibiting malignant transformation.
   Aspect 12. The method of any one of aspects 1-11 wherein said amount is effective to modulate said tumor by inhibiting metastasis.
   Aspect 13. The method of any one of aspects 1-12 wherein said amount is effective to modulate said tumor by reducing the number of circulating metastatic seed cells.
   Aspect 14. The method of any one of aspects 1-8, wherein the tumor is a benign tumor.
   Aspect 15. The method of aspect 14, wherein the benign tumor is a mass of precancerous cells.
   Aspect 16. The method of aspect 15, wherein the benign tumor is a mass of precancerous skin cells.
   Aspect 17. The method of aspect 15, wherein the benign tumor is a mass of precancerous cells of the respiratory tract, preferably, cells of the nasal cavity, paranasal sinuses, pharynx, larynx; trachea, bronchi, bronchioles, or lungs.
   Aspect 18. The method of aspect 15, wherein the benign tumor is a mass of precancerous cells of the digestive tract, preferably, cells from mouth, throat; esophagus; stomach; small intestine; colon; rectum, or anus.
   Aspect 19. The method of aspect 15, wherein the benign tumor is a mass of precancerous cells of the genitourinary tract organs, preferably, cells from kidney, bladder, prostate, testicles, uterus, fallopian tubes, ovaries, vagina, or external genitalia.
   Aspect 20. The method of aspect 15, wherein the benign tumor is a mass of precancerous blood cells.
   Aspect 21. The method of aspect 15, wherein the benign tumor is a mass of precancerous breast cells.
   Aspect 22. The method of aspect 15, wherein the benign tumor is a mass of precancerous cells of the endocrine organs, preferably, cells of the hypothalamus; pineal gland; pituitary gland; thyroid; parathyroid; thymus; adrenal gland, or pancreas.
   Aspect 23. The method of any one of aspects 1-8, wherein the tumor is a malignant tumor.
   Aspect 24. The method of aspect 23, wherein the malignant tumor is a respiratory tract tumor, preferably, of the nasal cavity, paranasal sinuses, pharynx, larynx; trachea, bronchi, bronchioles, or lungs.
   Aspect 25. The method of aspect 23, wherein the malignant tumor is a digestive tract tumor, preferably, of the mouth, throat; esophagus; stomach; small intestine; colon; rectum, or anus.
   Aspect 26. The method of aspect 23, wherein the malignant tumor is a genitourinary tract tumor, preferably, of the kidney, bladder, prostate, testicles, uterus, fallopian tubes, ovaries, vagina, or external genitalia.
   Aspect 27. The method of aspect 23, wherein the malignant tumor is cancerous blood cells.
   Aspect 28. The method of aspect 23, wherein the malignant tumor is breast tumor.
   Aspect 29. The method of aspect 23, wherein the malignant tumor is an endocrine tumor, preferably, of the hypothalamus; pineal gland; pituitary gland; thyroid; parathyroid; thymus; adrenal gland, or pancreas.
   Aspect 30. The method of aspect 23, wherein the malignant tumor is apocrine gland adenocarcinoma of the anal sac, hemangiosarcoma, lymphoma, mammary tumor, mast cell tumor, melanoma, oral squamous cell carcinoma, osteosarcoma, appendicular osteosarcoma, soft tissue sarcoma, solar induced squamous cell carcinoma, transitional cell carcinoma, a vaccine-associated sarcoma, adrenal medullary tumor, apocrine gland tumor, chondrosarcoma, esophageal cancer, exocrine pancreatic cancer, gastric cancer, hemangiosarcoma, hepatobiliary tumor, hyperadrenocorticism, intestinal tumor, intracranial neoplasia, larynx and trachea cancer, lymphoid leukemia, malignant histiocytoma, myelodysplasia, acute myeloid leukemia, myeloproliferative diseases, nasal chondrosarcoma, nasosinal tumor, nerve sheath tumor, ovarian tumor, plasma cell neoplasia, prostate cancer, pulmonary tumors, rhabdomyosarcoma, salivary gland cancer, sebaceous and modified sebaceous gland tumor, spinal cord neoplasia, testicular tumor, thyroid gland neoplasia, urinary bladder cancer, uterine tumor, vaginal tumor, or vulvar tumor.
   Aspect 31. The method of any one of aspects 1-30 wherein the tyrosine derivative is administered orally, subcutaneously, intravenously, transdermally, vaginally, rectally or in any combination thereof.
   Aspect 32. The method of any one of aspects 1-31, further comprising administering an effective amount of one or more additional therapeutic agents.
   Aspect 33. The method of aspect 32, wherein the additional therapeutic agent is 5 fluorouracil, actinomycin, bleomycin, carboplatin, chlorambucil, cisplatin, cyclophosphamide, cyclophosphamine, cytosine arabinoside, doxorubicin, dacarbazine (DTIC), L-asparaginase, melphalan, methotrexate, mitoxantrone, piroxicam, prednisone, vinblastine, or vincristine.
   Aspect 34. The method of any one of aspects 1-33 wherein the non-human mammal is a dog, cat, horse, cow, pig, monkey, rabbit, lamb, mouse, or goat.
   Aspect 35. The method of any one of aspects 1-34, wherein the method results in reduction or elimination of pain caused by said tumor.

1. A method for treating a non-human mammal that has a benign tumor or a malignant tumor, comprising administering to said mammal an amount of at least one tyrosine derivative that is effective to modulate said tumor.

2. A method for treating a non-human mammal that has a benign tumor or a malignant tumor, comprising administering to said mammal an amount of a combination of at least one tyrosine derivative and one or more of: (a) melanin, a melanin promoter, or a combination thereof; (b) at least one p450 3A4 promoter; (c) a leucine aminopeptidase inhibitor; (d) growth hormone inhibitor; and (e) testosterone or a derivative thereof; wherein said amount is effective to modulate said tumor.

3. The method of claim 2 wherein the tyrosine derivative is one or more of:
methyl (2R)-2-amino-3-(2-chloro-4-hydroxyphenyl) propanoate;
D-tyrosine ethyl ester hydrochloride;
methyl (2R)-2-amino-3-(2,6-dichloro-3,4-dimethoxyphenyl) propanoate;
H-D-Tyr(TBU)-allyl ester HCl;
methyl (2R)-2-amino-3-(3-chloro-4,5-dimethoxyphenyl) propanoate;
methyl (2R)-2-amino-3-(2-chloro-3-hydroxy-4-methoxyphenyl) propanoate;
methyl (2R)-2-amino-3-(4-[(2-chloro-6-fluorophenyl) methoxy] phenyl) propanoate;
methyl (2R)-2-amino-3-(2-chloro-3,4-dimethoxyphenyl) propanoate;
methyl (2R)-2-amino-3-(3-chloro-5-fluoro-4-hydroxyphenyl) propanoate;
diethyl 2-(acetylamino)-2-(4-[(2-chloro-6-fluorobenzyl) oxy] benzyl malonate;
methyl (2R)-2-amino-3-(3-chloro-4-methoxyphenyl) propanoate;
methyl (2R)-2-amino-3-(3-chloro-4-hydroxy-5-methoxyphenyl) propanoate;
methyl (2R)-2-amino-3-(2,6-dichloro-3-hydroxy-4-methoxyphenyl) propanoate;
methyl (2R)-2-amino-3-(3-chloro-4-hydroxyphenyl) propanoate;
H-DL-tyr-OMe HCl;
H-3,5-diiodo-tyr-OME HCl;
H-D-3,5-diiodo-tyr-OME HCl;
H-D-tyr-OMe HCl;
D-tyrosine methyl ester hydrochloride;
D-tyrosine-OMe HCl;
methyl D-tyrosinate hydrochloride;
D-tyrosine methyl ester HCl;
H-D-Tyr-OMe-HCl;
(2R)-2-amino-3-(4-hydroxyphenyl) propionic acid;
(2R)-2-amino-3-(4-hydroxyphenyl) methyl ester hydrochloride;
methyl (2R)-2-amino-3-(4-hydroxyphenyl) propanoate hydrochloride;
methyl (2R)-2-azanyl-3-(4-hydroxyphenyl) propanoate hydrochloride;
3-chloro-L-tyrosine;
3-nitro-L-tyrosine;
3-nitro-L-tyrosine ethyl ester hydrochloride;
DL-m-tyrosine;
DL-o-tyrosine;
Boc-Tyr (3,5-I2)-OSu;
Fmoc-tyr(3-NO2)-0H;
α-methyl-L-tyrosine;
α-methyl-D-tyrosine;
α-methyl-DL-tyrosine; and
C₁-C₁₂ alkylester salts of α-methyl-DL-tyrosine such as α-methyl-DL-tyrosine methyl ester hydrochloride.

4. The method of claim 3 wherein the tyrosine derivative is α-methyl-DL-tyrosine.

5. The method of claim 2, wherein said mammal is administered an amount of a combination of at least one tyrosine derivative and (a) melanin, a melanin promoter, or a combination thereof; (b) at least one p450 3A4 promoter; (c) a leucine aminopeptidase inhibitor; and (e) testosterone or a derivative thereof, wherein said amount is effective to modulate said tumor.

6. The method of claim 5, wherein the tyrosine derivative is α-methyl-DL-tyrosine; (a) is melanin, methoxsalen, melanotan, or melanotan II; (b) is 5,5-diphenylhydantoin; (c) Bestatin, rapamycin, or everolimus, and (e) is dihydrotestosterone.

7. The method of claim 6, wherein the tyrosine derivative is α-methyl-DL-tyrosine; (a) is melanotan II; (b) is 5,5-diphenylhydantoin; (c) Bestatin, and (e) is dihydrotestosterone.

8. The method of claim 6, wherein the tyrosine derivative is α-methyl-DL-tyrosine; (a) is melanotan (b) is 5,5-diphenylhydantoin; (c) is Bestatin, and (e) is dihydrotestosterone.

9. The method of claim 2, wherein said amount is effective to modulate said tumor by
reducing cell proliferation in the tumor;
reducing the rate of tumor growth;
inhibiting malignant transformation;
inhibiting metastasis; or
reducing the number of circulating metastatic seed cells.

10.-13. (canceled)

14. The method of claim 2, wherein the tumor is a benign tumor.

15. The method of claim 14, wherein the benign tumor is a mass of precancerous cells.

16. The method of claim 15, wherein the benign tumor is a mass of precancerous skin cells;
a mass of precancerous cells of the respiratory tract, nasal cavity, paranasal sinuses, pharynx, larynx, trachea, bronchi, bronchioles, or lungs;
a mass of precancerous cells of the digestive tract, mouth, throat; esophagus; stomach; small intestine; colon, rectum, or anus;
a mass of precancerous cells of the genitourinary tract organs, kidney, bladder, prostate, testicles, uterus, fallopian tubes, ovaries, vagina, or external genitalia;
a mass of precancerous blood cells; a mass of precancerous breast cells; or
a mass of precancerous cells of the endocrine organs, hypothalamus, pineal gland; pituitary gland, thyroid, parathyroid, thymus, adrenal gland, or pancreas.

17.-22. (canceled)

23. The method of claim 2, wherein the tumor is a malignant tumor.

24. The method of claim 23, wherein the malignant tumor is
a respiratory tract tumor; a respiratory tract tumor of the nasal cavity, paranasal sinuses, pharynx, larynx, trachea, bronchi, bronchioles, or lungs;
a digestive tract tumor; a digestive tract tumor of the mouth, throat, esophagus, stomach, small intestine, colon, rectum, or anus;
a genitourinary tract tumor; a genitourinary tract tumor of the kidney, bladder, prostate, testicles, uterus, fallopian tubes, ovaries, vagina, or external genitalia;
cancerous blood cells;
breast tumor; or
an endocrine tumor; an endocrine tumor of the hypothalamus, pineal gland, pituitary gland, thyroid, parathyroid, thymus, adrenal gland, or pancreas.

25.-29. (canceled)

30. The method of claim 23, wherein the malignant tumor is apocrine gland adenocarcinoma of the anal sac, hemangiosarcoma, lymphoma, mammary tumor, mast cell tumor, melanoma, oral squamous cell carcinoma, osteosarcoma, appendicular osteosarcoma, soft tissue sarcoma, solar induced squamous cell carcinoma, transitional cell carcinoma, a vaccine-associated sarcoma, adrenal medullary tumor, apocrine gland tumor, chondrosarcoma, esophageal cancer, exocrine pancreatic cancer, gastric cancer, hemangiosarcoma, hepatobiliary tumor, hyperadrenocorticism, intestinal tumor, intracranial neoplasia, larynx and trachea cancer, lymphoid leukemia, malignant histiocytoma, myelodysplasia, acute myeloid leukemia, myeloproliferative diseases, nasal chondrosarcoma, nasosinal tumor, nerve sheath tumor, ovarian tumor, plasma cell neoplasia, prostate cancer, pulmonary tumors, rhabdomyosarcoma, salivary gland cancer, sebaceous and modified sebaceous gland tumor, spinal cord neoplasia, testicular tumor, thyroid gland neoplasia, urinary bladder cancer, uterine tumor, vaginal tumor, or vulvar tumor.

31. The method of claim 2 wherein the tyrosine derivative is administered orally, subcutaneously, intravenously, transdermally, vaginally, rectally or in any combination thereof.

32. The method of claim 2, further comprising administering an effective amount of one or more additional therapeutic agents.

33. The method of claim 32, wherein the additional therapeutic agent is 5 fluorouracil, actinomycin, bleomycin, carboplatin, chlorambucil, cisplatin, cyclophosphamide, cyclophosphamine, cytosine arabinoside, doxorubicin, dacarbazine (DTIC), L-asparaginase, melphalan, methotrexate, mitoxantrone, piroxicam, prednisone, vinblastine, or vincristine.

34. The method of claim 2 wherein the non-human mammal is a dog, cat, horse, cow, pig, monkey, rabbit, lamb, mouse, or goat.

35. The method of claim 2, wherein the method results in reduction or elimination of pain caused by said tumor.
